(19) United States
(12) Patent Application Publication
(10) Pub. No.: US 2007/0160663 A1
(43) Pub. Date: Jul. 12, 2007

(54) Single unit pharmaceutical composition comprising a mixture of fenofibrate and a modified release form of a homocysteine reducing agent

(75) Inventors: Arthur M. Deboeck, Gurabo, PR (US); Philippe Baudier, Uccle (BE); Francis Vanderbist, Beersel (BE); Antonio Sereno, Melsbroeck (BE)

Correspondence Address: MERCHANT & GOULD PC, P.O. BOX 2903, MINNEAPOLIS, MN, 55402-0903, US

(73) Assignee: GALEPHAR PHARMACEUTICAL RESEARCH, INC.

(21) Appl. No.: 10/567,407

(22) PCT Filed: Aug. 4, 2004
(86) PCT No.: PCT/BE04/00111

§ 371 Date: Nov. 13, 2006

(30) Foreign Application Priority Data
Aug. 5, 2003 (BE) PCT/BE03/00132

(51) Int. Cl. A61K 31/525; A61K 31/192; A61K 9/64; A61K 9/22

(52) U.S. Cl. 424/456; 514/571; 514/251; 424/468

(57) ABSTRACT

An oral lipid lowering pharmaceutical unit form comprising a first solid or semi solid composition comprising fenofibrate and a second solid or semi solid composition comprising at least a modified release form of an homocysteine lowering agent.

The present invention relates to a single unit pharmaceutical composition comprising fenofibrate and at least a homocysteine lowering agent useful to reduce the plasma levels of homocysteine in patients to whom fibrates are administered. The homocysteine lowering agent is selected from the group consisting of folic acid, vitamin B12, Vitamin B6 and Betaine. The homocysteine lowering agent is formulated as a modified release composition i.e. delayed or extended release

The composition of the present invention may be administered to patients once a day.

Compositions of the present invention are further characterised that the single dosage unit contains amounts of fenofibrate comprised between 25 mg and 300 mg and therapeutic amounts of one or several modified release homocysteine lowering agents

### BACKGROUND OF THE INVENTION

Number of studies have shown that the lipid lowering therapy including administration of drugs from the fibrate family is associated with an increase of the plasma concentration of total homocysteine. While more studies are needed that investigate the underlying mechanism responsible for the homocysteine increase, it appears that such increases of homocysteine are associated with increased cardiovascular risks and increase incidence of cerebro-vascular diseases.

The increase of homocysteine in patients is known as hyperhomocysteinemia and can be divided into three classes: Severe (homocysteine plasma concentration >100 μmol/L), moderate (25 to 100 μmol/L), or mild (16 to 24 μmol/L).

Severe hyperhomocysteinemia is usually caused by a homozygous deficiency of the enzyme cystathionine β-synthase. Affected persons have severe mental retardation, ectopic lens, skeletal abnormalities, and severe premature arterial and venous thrombotic disease.

Mild or moderate hyperhomocysteinemia is found in patients with either hereditary or acquired defects in the homocysteine metabolic pathway. Heterozygous deficiency in cystathionine β-synthase is quite common in the general population, with a frequency of 0.3% to 1.4%. A defect in the remethylation pathway is commonly caused by a thermolabile mutant of the methylene-tetra-hydofolate reductase (MTHFR) enzyme that has approximately 50% of the normal enzyme activity; the homozygous state has a prevalence of 5% in the general population. Common causes of acquired hyperhomocysteinemia are deficiency of dietary cobalamin, folate, or pyridoxine (the essential cofactors for the homocysteine metabolic pathway). A recent prospective study showed that mild hyperhomocysteinemia is quite common in the elderly, despite normal serum vitamin concentrations.

Mild to moderate hyperhomocysteinemia is associated with cerebrovascular disease, coronary artery disease, and peripheral vascular disease in persons younger than 55 years and with carotid artery stenosis in the elderly. It is found in 10% of patients with a first episode of DVT (Deep Vein Thrombosis). In a recent prospective study, a graded relationship was found between elevated plasma homocysteine levels and mortality in patients with coronary artery disease.

Homocysteine is a highly reactive amino acid containing a free sulfhydryl group. It can promote oxidation of low-density lipo-protein (LDL) cholesterol and presumably is toxic to vascular endothelium. It may also inhibit thrombomodulin expression and protein C activation and suppress endothelial heparan sulfate expression, both of these effects lead to hypercoagulability. Recently, homocysteine was shown to enhance the binding of lipoprotein(a), and atherogenic lipoprotein to fibrin, which may provide a link between hyperhomocysteinemia, thrombosis, and premature atherosclerosis. The vascular damage caused by high homocysteine levels leads to arterial and venous thrombosis and, perhaps, accelerated atherosclerosis.

Fenofibrate pertain to the lipid lowering family drugs of fibrates.

The lipid-modifying effects of fenofibrate are mediated via the activation of the peroxisome proliferator-activated receptors (PPARs).

Fenofibrate reduce plasma total cholesterol (TC), low-density lipoprotein cholesterol (LDL-C), triglyceride (TG) and very-low-density lipoprotein (VLDL) cholesterol levels, and increase high-density lipoprotein cholesterol (HDL-C) and apolipoprotein (Apo) AI and Apo AII levels in patients with dyslipidaemia. Fenofibrate also reduce plasma fibrinogen levels in both normolipidemic individuals and those with dyslipidemia, and is significantly more effective in that reduction than Simvastatin, Atorvastatin or Pravastatin. This is of significance since increased levels of fibrinogen or plasminogen activator inhibitor (PAI-1) are associated with an increased risk of atherosclerosis and coronary heart disease (CHD).

Fenofibrate has also demonstrated a very important activity in reducing the levels of the inflammatory marker C reactive protein (CRP), which has been recognized to have a negative effect on the evolution of the pathogenesis of atherosclerosis and coronary heart diseases.

Fenofibrate or p-(4-chlorobenzoyl)-phenoxy isobutyrate isopropyl ester is useful for the treatment of adult patients with very high elevations of serum triglyceride levels and/or cholesterol levels. The usual daily dosage is 50 to 300 mg which is administered in one or two doses. Fenofibrate absorbed as fenofibric acid, resulting from the hydrolysis of fenofibrate, is extensively bound to plasma albumin. Fenofibric acid has a Tmax (time to peak plasma level) of 3-6 hours after oral administration of a conventional marketed form such as Lipanthyl® tablet or Fenogal® capsule.

The plasma half-life is about 20 hours. Fenofibric acid is excreted predominantly in the urine, mainly as the glucuronide conjugate, but also as a reduced form of fenofibric acid and its glucuronides.

While some study results seem to be contradictory. It is now, commonly admitted by the medical community that, Fenofibrate administration (like other fibrates) to patient increases the level of homocysteine in the plasma (Drug Safety 2003:26 (2) 81-91).

The effect of Fenofibrate, compared with Placebo on total plasma homocysteine levels in the fasted and the fed states has been examined. Fenofibrate caused marked decrease in all triglyceride rich protein parameters and was associated with an increase in fasting total homocysteine, from 10.3 μmol/L to 14.4 μmol/L (+40%) and fed total homocysteine levels 6 hours past prandil load from 11.6 μmol/L t0 17.1 μmol/L [Atherosclerosis. 2001 April: 155(2): 455-62).

A homocysteine lowering agent is defined as a substance able to decrease plasma levels of homocysteine in humans in such a need. Examples of those homocysteine lowering agents are: Folic acid, vitamin B6, vitamin B12 and Betaine.

Also it has been shown that in patient not receiving lipid-lowering drugs, vitamin supplementation with folic acid and vitamin B12 effectively reduces the plasma homocysteine levels.

Also, while some studies have shown that folic acid or vitamin combination to Fenofibrate could allow to decrease the homocysteine increase associated with the Fenofibrate administration. These studies were performed alternately (one day fibrate and one day vitamin) or by administration of folic acid, vitamin B6 and/or B12 upon completion of the fibrate treatment or by a separate administration of fenofibrate and the lipid lowering agent. Also the lipid lowering agent was always given as an immediate release oral form what is probably not the most efficient way of administration.

What was never disclosed, nor suggested is an oral single unit pharmaceutical composition consisting of the combination of a therapeutic effective amount of fibrate derivative with at least an effective amount of lowering homocysteine agent or a mixture of such lowering homocysteine agents, the release of this homocysteine lowering agent(s) being controlled in order to better suit to the release of fenofibrate.

By controlled release composition, we mean any composition which is not an immediate release composition (also called conventional form). In other words controlled release compositions are compositions containing an agent being capable of modifying the release of the compound (when compared to immediate release forms) either by delaying it or by prolonging it. Examples of such formulations are coated tablets or matrix tablets, coated or matrix beads, osmotic pumps, bioadhesive forms, multilayer tablets, fatty matrix, . . .

In the present invention the terms “beads”, “pellets” and “microgranules” are synonyms

Indeed, as fenofibrate present a Tmax of 3-6 hours after oral administration and has a long half life, it would be advantageous to release the homocysteine lowering agent(s) in such a way that its tmax is close to the one of fenofibrate. Pharmaceutical compositions containing folic acid, vitamin B12 and vitamin B6 of mixtures thereof are available on the market as immediate release forms i.e. forms releasing the compound immediately in the gastro-intestinal tract. Although the value of Tmax of those substances may vary depending on the compound considered, the tmax is often of 0.5 to 2 hours after oral administration.

The object of the present invention is to dispose, in the same pharmaceutical form, of a combination of fenofibrate and an homocysteine lowering agent, said form releasing fenofibrate in a similar way as the compositions of fenofibrate available on the market (Tmax of 3-6 hours) and the homocysteine lowering agent in a modified release in order be as close as possible to fenofibrate's release (Tmax at least greater than 1 hourn preferably greater than 2 hours).

A single unit form is a pharmaceutical form containing both the fibrate derivative and the modified release homocysteine lowering agent in such a way that the patient can swallow the said pharmaceutical form in a single intake.

Also, all the previous art was directed towards reducing the levels of homocysteine after they were first increased while an object of the present invention is to provide for a pharmaceutical composition that avoids the increase of homocysteine in the patient. In other words, the present invention relates to a pharmaceutical composition containing a fibrate and able, to some extent, to prevent the increase of homocysteine plasma levels caused by the fibrate.

It is an object of the present invention to provide an orally administered pharmaceutical composition of a fibrate and a modified release homocysteine lowering agent that provides for a therapeutically effective amount of the fibrate and that substantially reduces the increase of plasma homocysteine otherwise encountered after administration of such amount of fibrate to the patient.

It is another object of the present invention to provide an orally administered pharmaceutical composition of a fibrate and a modified release homocysteine lowering agent which is contained into a single unit formulation.

It is another object of the present invention to provide an orally administered pharmaceutical composition of a fibrate and a modified release homocysteine lowering agent which is suitable for once a day administration.

It is another object of the present invention to provide an orally administered pharmaceutical composition of a fibrate and a modified release homocysteine lowering agent, from which the release of the homocysteine lowering agent is delayed, extended or any combination for thereof.

It is another object of the present invention to provide an orally administered pharmaceutical composition of a fibrate and a homocysteine lowering agent which comprises a modified release homocysteine lowering agent selected from the group comprising folic acid, vitamin B6, vitamin B12, betaine alone or in mixtures thereof.

It is another object of the present invention to provide a method of treatment of hypercholesterolemia and related diseases of dyslipidemia comprising the administration of the dosage forms of the composition of the present invention to a patient in need of treatment.

Details and advantageous characteristics of compositions of the invention are given in the attached claims.

### DETAILED DESCRIPTION OF THE INVENTION

Different pharmaceutical formulations may be used to obtain the single unit form of the present invention. For instance, a capsule containing a coated, or uncoated or multilayer tablet of a modified release homocysteine lowering agent with a semi-solid composition of fenofibrate is suitable.

Other alternatives are capsules containing the modified release homocysteine lowering agent under the form of pellets or tablets and fenofibrate formulated as a paste, semi-solid tablet, granulated powder or pellets, coated or uncoated tablets, but always combined in a single unit form.

The modified release form of homocysteine lowering agent release may be a delayed form such as an enteric tablet or capsule, or a sustained release form (tablet or granules) or a form combining an immediate release form of the homocysteine lowering agent with a prolonged release form of the same homocysteine lowering agent.

The homocysteine lowering agent may also be present in the final composition as bilayer tablet where the homocysteine lowering agent is in the core (central layer or inner layer) of the tablet and the sustained release properties are conferred by the outer layer of the tablets. This formulation presents the advantage to avoid any physical contact between the homocysteine lowering agent and fenofibrate, and hence to prevent any kind of chemical interaction between the two compounds. Furthermore, this composition can enhance the stability properties of the homocysteine lowering agent within the final composition

As fenofibrate present a relatively long elimination half-life, from 20 to 90 hours and some of vitamin B derivatives present a short half-life (folic acid: 3 hours), it is particularly advantageous for the present invention to provide the patient with a composition where the fibrate is formulated as an immediate release form and the vitamin B derivative at least partly as a sustained or delayed release formulation (both derivatives being finally put into a single unit form) in order to optimize the duration of action of the vitamin B derivative and to increase as much as possible its therapeutic homocysteine lowering effect.

For instance, the single unit final form can be a capsule containing a semi-solid formulation of fenofibrate, and a sustained release tablet (coated or not) containing the vitamin B derivative.

Examples of such sustained release vitamin B formulations can be matrix tablets containing an hydrophilic or an hydrophobic polymer (or a mixture thereof), bilayer or multilayer tablets, sustained release coated granules, matrix granules, etc. . . .

When formulating sustained release compositions of vitamins B derivatives, the absorption window should be taken into account. For instance, folic acid and vitamin B6 have their main absorption window in the proximal jejunum. The sustained release of folic acid or vitamin B6 should therefore not be too slow because it should be delivered completely within the absorption window to keep an acceptable bioavailability. For instance, such sustained release formulations of folic acid should present a Tmax in vivo of between 1 and 10 hours, preferably between 2 and 8 hours, more preferably between 2 and 6 hours. When tested in vitro, on a paddle dissolution apparatus (EP 2003, 4^{th} edition, 2.9.3) at 100 round per minute (rpm) in water, the dissolution rate is for instance of 0 to 50% after 30 minutes, 5 to 75% dissolved after 1 hour, 20 to 90% dissolved after 2 hours, 50-95% dissolved after 4 hours and more than 80% dissolved after 8 hours.

Alternatively the homocysteine lowering agent can be a combination of various homocysteine lowering agents such as, but not limited to, a combination of folic acid and vitamin B12 or a combination of folic acid and vitamin B6 or even a combination of folic acid with vitamin B12 and vitamin B6. In this case, all of the homocysteine lowering agents are present as modified release forms or alternatively some of the homocysteine lowering agents are present as modified release forms and other are immediate release.

Also alternatively, the homocysteine lowering agent can be formulated in such a way that it presents a biphasic or multiphasic release what means that it, for instance, can present both immediate and sustained release properties. For instance, a sustained release matrix tablet of folic acid may be further coated with an additional amount of folic acid (which is release rapidly). This coated tablet is then put into a capsule with a semi-solid composition of fenofibrate to obtain the final composition of the present invention.

### EXAMPLES

The invention is additionally illustrated in connection with the following examples, which are considered to be illustrative of the present invention. It should be understood, however, that the invention is not limited to the specific details of the Examples.

### Example 1

### Example 1 a

### Folic Acid Uncoated Tablet

| Ingredient Name | Amount [g] |
|---|---|
| folic acid | 1 |
| Lactose monohydrate | 100 |
| Cellulose microcrystalline | 36 |
| Povidone K30 | 2 |
| Water for granulation | 25 |
| Magnesium stearate | 2 |
| Sodium starch glycolate | 13 |

Folic acid, lactose monohydrate, cellulose microcrystalline and povidone K30 were blended in a planetary mixer for about 5 to 10 minutes until an homogeneous blend is obtained. While under agitation, a solution containing the water for granulation is added to granulate the powders. The granules obtained are dried at about 40° C. for about 5 hours. Thereafter the dried granules are screened through a 1.0 mm sieve, and further blended into a planetary mixer for about 2 minutes after the addition of the magnesium stearate and sodium starch glycolate.

The final mix is compressed into tablets using a rotary compressing machine equipped with punches of the deep cup type with a diameter of 6.5 mm. The mean weight of the tablets is of about 180 mg, corresponding to tablets containing 1 mg of folic acid. The tablet hardness is comprised between 4 and 6 kilopascals (Kp).

### Example 1b

### Coating of Folic Acid Tablets

| Ingredient Name | Amount [g] |
|---|---|
| Povidone K30 | 15 |
| ethylcellulose | 5 |
| Talc | 35 |
| Triacetin | 5 |
| Absolute Alcohol | 300 |

The coating solution of example 1b is applied to the tablets from Example 1a using a pan coater. The amount of coating applied is about 15 mg of dry coating (weight gain) per tablet. In this composition, ethylcellulose is the agent responsible of the prolonged release of folic acid.

### Example 1 c

### Semi-Solid Fenofibrate Composition

| Ingredient Name | Amount [g] |
|---|---|
| Fenofibrate powder | 160 |
| Lauroyl macrogolglyceride | 240 |
| (gelucire 44/14) | |
| Polyethylene glycol 20,000 | 48 |
| Hydroxypropylcellulose | 95.0 |
| Sodium starch glycolate | 20.0 |
| Ascorbyl palmitate | 1.0 |

Gelucire 44/14 and polyethylene glycol 20,000 are added to a mixer equipped with a double wall bowl. The mixer is started and the bowl is warmed at about 75° C. When the gelucire and the polyethylene glycols are molten, the other ingredients (Fenofibrate, hydroxypropyl cellulose, sodium starch glycolate and ascorbyl palmitate) are added while maintaining the temperature at about 70-75° C.

### Example 1

### Final Composition

The combination product is obtained by filling, into size 0 elongated hard gelatin capsules, one tablet of Example 1a coated with 1 b and 564 mg of the hot blend of Example 3. After filling, the capsules are cooled by placing them on trays between 4 and 8° C.

The capsules obtained contained 1 mg of folic acid and 160 mg of fenofibrate. It should be noted that the matrix tablet obtained allow to deliver folic acid in a sustained release manner as demonstrated by the in vitro dissolution curve given hereinbelow.

In Vitro Dissolution Curve of Folic Acid And Fenofibrate From the Final Composition of Example 1 In Comparison With A Marketed Form of Folic Acid (Folavit®, Wolfs)(Paddle Apparatus, 100 Rpm, Phosphate Buffer 7.5)

[image]

### Example 2

### Example 2a

### Folic Acid And Vitamin B12 Matrix Tablets

| Ingredient Name | Amount [g] |
|---|---|
| folic acid | 2 |
| vitamin B12 | 0.5 |
| EUDRAGIT ® NE30D | 10 |
| Lactose monohydrate | 100 |
| Cellulose microcristalline | 40 |
| Povidone K30 | 2 |
| Water for granulation | 30 |
| Magnesium stearate | 2 |
| Sodium starch glycolate | 13 |

Folic acid, Vitamin B12, Lactose monohydrate, cellulose microcrystalline and povidone K30 are blended in a planetary mixer for about 5 to 10 minutes until an homogeneous blend is obtained. While under agitation, an aqueous suspension of EUDRAGIT® NE30D (polyacrylate dispersion 30% which is the agent responsible for the controlled release) into the water for granulation is added to granulate the powders. The granules obtained are dried at about 40° C. for about 5 hours. After the dried granules are screened through a 1.0 mm sieve, they are blended into a planetary mixer for about 2 minutes after the addition of the magnesium stearate and sodium starch glycolate. The final mix is compressed into tablets using a rotary compressing machine equipped with punches of the deep cup type with a diameter of 6.5 mm. The mean weight of the tablets is of about 200 mg. The tablets had hardness comprised between 4 and 6 kilopascals (Kp).

### Example 2b

### Fenofibrates Granules

| Ingredient | Amount [g] |
|---|---|
| Fenofibrate powder | 160 |
| Lactose | 300 |
| Povidone K30 | 15 |
| Sodium Lauryl Sulfate | 7 |
| Crospovidone | 15 |
| Magnesium Stearate | 3 |

Fenofibrate, lactose, povidone and sodium lauryl sulfate are blended in a planetary mixer and water is added to granulate. After oven drying for about 5 hours at 50° C., the granules are screened through a 1 mm sieve. After addition of crospovidone and the magnesium stearate the granules that are blended for an additional 3 minutes in the planetary mixer.

### Example 2

### Final Composition

500 mg of lubricated granules of Example 2b and a tablet of Example 2a are filled into 0 elongated hydroxypropylmethylcellulose capsules to produce a combination product containing 2 mg of folic acid, 0.5 mg of vitamin B12 and 160 mg of fenofibrate.

### Example 3

### Example 3a

### Folic Acid Coated Beads

| Ingredient Name | Amount [g] |
|---|---|
| Folic acid | 5 |
| sucroester (Crodesta ®) | 20 |
| Microcrystalline cellulose | 100 |
| Povidone K30 | 20 |
| | 145 |

Water is added to the blend of all the ingredients in a planetary mixer to granulate the powder. The paste obtained is extruded and spheronized in order to obtain beads with a diameter of about 1 mm. The beads are tray dried in an oven at about 40° C. for approximately 5 hours. The beads are thereafter screened between 0.7 mm and 1.4 mm sieves.

500 g of beads from Example 3a are coated with 200 g of coating solution) of Example 1b using a fluid bed coater (Strea 1) equipped with a wurster column.

### Example 3

### Final Composition

A combination formulation is produced by filling in a 00 hard gelatin capsules with 500 mg of Fenofibrate lubricated granules of Example 2b and 145 mg of folic acid beads of Example 3a.

The resulting combination formulation contained 5 mg of folic acid and 160 mg of fenofibrate.

### Example 4

### Bilayer Tablets

### Example 4a

Inner Layer Containing Folic Acid

| Ingredient Name | Amount [mg]/tablet |
|---|---|
| Folic acid | 5 |
| aerosil ® | 0.2 |
| Microcrystalline cellulose | 5 |
| Mannitol | 19.31 |
| Magnesium stearate | 0.48 |
| Butylhydroxyanisole | 0.01 |

The diameter of the inner tablet is 4 mm and the hardness around 2 Kp

### Example 4b

### Outer Layer (Sustained Release Layer)

| Ingredient Name | Amount [mg]/tablet |
|---|---|
| Lactose | 62 |
| Mannitol | 33 |
| Stearic acid | 5 |
| Povidone | 5 |
| Magnesium stearate | 0.5 |

The diameter of the inner tablet is 6 mm and the hardness around 28-10 Kp

The bilayered tablet is obtaining by proceeding to the compression of the inner tablet and hence to proceed to the compression of the outlayer tablet around the inner tablet while maintaining the inner tablet centrally such as after compression of the outer tablet, the inner tablet is no more visible.

The final composition of example 4 is obtained by combining in a size 0 elongated hard gelatin capsule, 564 mg of the semi-solid composition of fenofibrate of example 1c with the bilayer tablets containing folic acid described hereinabove

The dissolution curve hereinbelow shows the extended release profile of the bilayer folic acid tablets in comparison to the core only. The bilayer tablets clearly possess extended release properties due to the presence of stearic acid in the outer layer of the tablet.

Comparative In Vitro Dissolution Curve of the Inner Layer Tablet (Core) Containing Folic Acid Alone And the Final Bilayer Extended Release Tablet (Paddle Apparatus, 100 Rpm, Phosphate Buffer 7.5)

[image]

What we claim is:

1. An oral lipid lowering pharmaceutical unit form comprising a first solid or semi solid composition comprising fenofibrate and a second solid or semi solid composition comprising at least a homocysteine lowering agent, the second composition being a modified release form

2. The pharmaceutical composition according to claim 1 characterised that it comprises an effective amount fenofibrate for the treatment of hyperlipidemia.

3. The pharmaceutical composition according to claim 1, in which the homocysteine lowering agent is selected from the group consisting of folic acid, vitamin B12, vitamin B6, Betaine, and mixtures thereof.

4. The pharmaceutical composition according to claim 1, in which the amount of Fenofibrate is comprised between 25 mg and 400 mg, preferably between 50 mg and 300 mg.

5. The pharmaceutical composition according to claim 1 in which the Fenofibrate is present in mixture with at least one polyglyceride.

6. The pharmaceutical composition according to claim 5 in which the Fenofibrate is present under the form of micronized Fenofibrate.

7. The pharmaceutical composition according to claim 1 from which the modified release of the homocysteine lowering agent is either delayed or extended or any combination of these releases.

8. The pharmaceutical composition according to claim 1 wherein the homocysteine lowering agent is folic acid.

9. The pharmaceutical composition of claim 1, in which the first composition is an substantially immediate release composition of fenofibrate.

10. The pharmaceutical composition according to claim 1, wherein the dose of fenofibrate is between 50 and 300 mg and the dose of the homocysteine lowering agent is between 0.001 and 100 mg.

11. The pharmaceutical composition according to claim 1 where the single unit form is a hard gelatin, hypromellose capsule or any other pharmaceutically acceptable capsule.

12. The pharmaceutical composition according to claim 1 where the single unit form is a tablet.

13. The pharmaceutical composition according to claim 1 wherein homocysteine lowering agent is an extended release form.

14. The pharmaceutical composition according to claim 1 wherein the second composition is a composition combining an immediate release form of a part of the homocysteine lowering agent with a prolonged release form of another part of the homocysteine lowering agent.

15. The pharmaceutical composition according to claim 1, in which the second composition is a composition controlling the release of the homocysteine lowering agent so as to ensure, after single dose administration of the composition to human volunteers a Tmax (time for reaching the maximum peak concentration in the human plasma) in vivo of between 1 and 10 hours, preferably between 2 and 8 hours, more preferably between 2 and 6 hours.

16. The pharmaceutical composition according to claim 1, in which the second composition is a composition controlling the release of the homocysteine lowering agent so as to ensure a dissolution rate in vitro, on a paddle dissolution apparatus (EP 2003, 4^{th} edition, 2.9.3) at 100 round per minute (rpm) in a 7.5 phosphate buffer, of 0 to 50% after 30 minutes, 5 to 75% after 1 hour, 20 to 95% after 2 hours, 50-95% after 4 hours and more than 80% after 8 hours.

17. The pharmaceutical composition according to claim 1 wherein the homocysteine lowering agent is a mix of two or more of said substances.

18. The pharmaceutical composition according to claim 1, wherein the final form is a capsule containing fenofibrate as a paste and folic acid as coated, uncoated or bilayer-modified release tablet.

19. The pharmaceutical composition according to claim 1, wherein the first solid or semi solid composition comprising a fibrate derivative is substantially free of homocysteine lowering agent and/or the second solid or semi solid composition comprising at least a homocysteine lowering agent is substantially free of fibrate derivative.

20. The pharmaceutical composition according to claim 18, further containing vitamin B12 in a modified release form

21. The pharmaceutical composition according to claim 14, further containing vitamin B12 in an extended release form
